(19) United States
(12) Patent Application Publication
(10) Pub. No.: US 2008/0154326 A1
(43) Pub. Date: Jun. 26, 2008

(54) METHOD AND APPARATUS FOR PROVIDING A RADIOPAQUE ANCHORING SLEEVE

(76) Inventor: Christopher A. Clyne, (US)

Correspondence Address: ST. ONGE STEWARD JOHNSTON & REENS, LLC, 986 BEDFORD STREET, STAMFORD, CT, 06905-5619, US

(21) Appl. No.: 11/614,671

(22) Filed: Dec. 21, 2006

(51) Int. Cl. A61N 1/05

(52) U.S. Cl. 607/36

(57) ABSTRACT

The invention relates to an anchoring sleeve for a lead of an implantable device, including a tube being of flexible material having a first end and a second end, the tube being adapted to permit the lead to extend from the first end to the second end, and where the tube also includes radiopaque material in at least one location.

### FIELD OF THE INVENTION

The invention relates to an anchoring sleeve for a lead of an implantable device.

### BACKGROUND OF THE INVENTION

Implantable leads are typically used in conjunction with various types of medical devices, such as pacemakers, cardiac defibrillators, neural stimulators, and the like. These leads are usually secured in some manner within the body, such as stitching or suturing, so that proper positioning and placement of the lead are maintained. However, stitching the lead in place may damage the lead, particularly over time where the stitch may sever the lead. When this occurs, the lead will normally need to be replaced, which typically entails surgery. Therefore, there is a need to reduce wear on the lead as this may also reduce unwanted surgeries.

One mechanism for securing the lead in a patient while also reducing wear on the lead may be to place an anchoring sleeve around the lead where the stitches are stitched directly to the sleeve, and indirectly to the lead. Because the sleeve is normally made of a flexible material, the sleeve may be compressed against the lead when the sleeve is stitched within the body, thereby resulting in the lead being secured. In this fashion, the lead may be protected by the anchoring sleeve yet secured in place.

Several examples of prior art suture sleeves are known in the prior art, including those disclosed in U.S. Pat. No. 4,516,584 issued on May 14, 1985 to Garcia entitled “Suture Collar” (cylindrical collar with longitudinal bore); U.S. Pat. No. 4,553,961 issued on Nov. 19, 1985 to Pohndorf et al. entitled “Suture Sleeve with Structure for Enhancing Pacing Lead Gripping” (cylindrical collar with longitudinal bore containing structure for enhancing gripping between collar and lead); U.S. Pat. No. 4,672,979 issued on Jun. 16, 1987 to Pohndorf entitled “Suture Sleeve Assembly” (tubular sleeve and collet member adapted to snap together); U.S. Pat. No. 4,683,895 issued on Aug. 4, 1987 to Pohndorf entitled “Suture Sleeve Anchoring Device” (circular staple-like clip for attaching a suture sleeve to tissue); U.S. Pat. No. 5,107,856 issued on Apr. 28, 1992 to Kristiansen et al. entitled “Multiple Lead Suture Sleeve” (generally “W”-shaped sleeve adapted to be compressed by sutures around one or two leads); and U.S. Pat. No. 5,129,405 issued to Milijasevic et al. on Jul. 14, 1992 entitled “Vein Suture Collar” (cylindrical collar with longitudinal bore).

Other tubular member securing mechanisms have been proposed in the prior art. Earlier examples include U.S. Pat. No. 3,176,690 issued on Apr. 6, 1965 to H'Doubler entitled “Catheter Having Integral, Polymeric Flanges” (elongated external flange integrally formed in the catheter body); U.S. Pat. No. 3,730,187 issued on May 1, 1973 to Reynolds (securing collar permanently located on the outer surface of the catheter and having a Dacron polyester suture embedded therein); and U.S. Pat. No. 3,724,467 issued on Apr. 3, 1973 to Avery et al. entitled “Electrode Implant for the Neuro-Stimulation of the Spinal Cord” (physiologically inert plastic tie-down clamp); which typically described various types of collars or tabs attached to the tubular member for providing a suturing structure.

Although the anchoring sleeve protects the lead from wear while permitting securement via the sutures, problems may arise with the sleeve when the lead is removed, where removal is usually the result of normal wear and tear on the lead or when the electrode on the lead fails. One problem may be locating the lead so that it may be removed. Another problem may be locating the sleeve so that it may be removed. A further problem may be infection or other complications when the lead or sleeve is accidentally left in the body due to an inability to locate them. This problem may become exacerbated when multiple leads and multiple sleeves are implanted in the body and the body's muscle and other tissue grow around and obscure visual contact with the lead. Also, when the lead is placed through the inner diameter of the sleeve, and where securement of the sleeve and lead is through sutures, the lead may be pulled out from the sleeve when the stitches are removed but the sleeve may be left behind. Therefore, locating the lead does not necessarily mean the sleeve is located as well.

Even if a sleeve is located, another problem may exist when the sleeve needs to be cut off in order to be removed, which sometimes occurs when the sleeve is buried or entangled in body tissue. By cutting the sleeve, there is a risk that the insulation of the lead or other fragments of the sleeve would be damaged and left behind, undetected due to their small size.

What is desired, therefore, is an anchoring sleeve that facilitates a surgical procedure by improving the extraction process of a lead that is coupled to both an implantable device and a chest wall or organ. Another desire is an anchoring sleeve that identifies its location within a body so that a doctor performing the surgical procedure may locate the sleeve and lead. A further desire is an anchoring sleeve that identifies its location for a prolonged period of time while simultaneously protecting the lead.

### SUMMARY OF THE INVENTION

It is, therefore, an object of the invention to provide a sleeve that is easily located once it is placed within the human body.

Another object is a sleeve that indicates its location and size so that a surgeon can easily find it and be confident in its complete removal.

A further object is a sleeve with radiopaque material located on the opposite ends of the sleeve for indicating a length of the sleeve.

Yet another object of the invention is a sleeve with radiopaque material located in several areas along a circumference of the sleeve for indicating a diameter of the sleeve.

These and other objects of the invention are achieved by an anchoring sleeve for a lead of an implantable device, including a tube being of flexible material having a first end and a second end, the tube being adapted to permit the lead to extend from the first end to the second end, and where the tube also includes radiopaque material in at least one location.

In some embodiments, the at least one location includes a localized area of said first end and said second end. In other embodiments, the at least one location includes a circumference of the first end and the second end. Optionally, the at least one location extends from said first end to said second end.

In some embodiments, the implantable device is a pacemaker and wherein the lead extends from the pacemaker and through the first and second ends. In other embodiments, the anchoring sleeve includes a stitch, wherein the tube compresses to permit the stitch to secure the lead to a chest wall or organ.

In another aspect of the invention, an anchoring sleeve for a lead of an implantable device includes a tube with an inner diameter, a device end, and a chest wall end. The tube is of a flexible material and adapted to receive the lead extending from the implantable device through the inner diameter and exiting the chest wall end. The chest wall end and the device end each includes a radiopaque material in a first location for indicating a length of the tube and the chest wall end and the device end each includes a radiopaque material in a second location for indicating a width of the tube.

In yet another aspect of the invention, an anchoring sleeve for a lead of an implantable device includes a pacemaker with a lead extending from the pacemaker and a tube having an inner diameter, a pacemaker end, and a chest wall end. The tube is of flexible material and adapted to permit the lead extend from the pacemaker end to the chest wall end, where the chest wall end and device end each includes a radiopaque material in a first location for indicating a length of the tube and the chest wall end and device end each includes a radiopaque material in a second location for indicating a width of the tube.

### BRIEF DESCRIPTION OF THE DRAWINGS

FIG. 1 depicts the anchoring sleeve in accordance with the invention.

FIG. 2 more particularly depicts the sleeve shown in FIG. 1.

FIG. 3 depicts a variation of the sleeve shown in FIG. 2.

FIG. 4 more particularly depicts the sleeve shown in FIG. 1.

FIG. 5 depicts a method or providing the sleeve shown in FIG. 1.

### DETAILED DESCRIPTION OF THE INVENTION

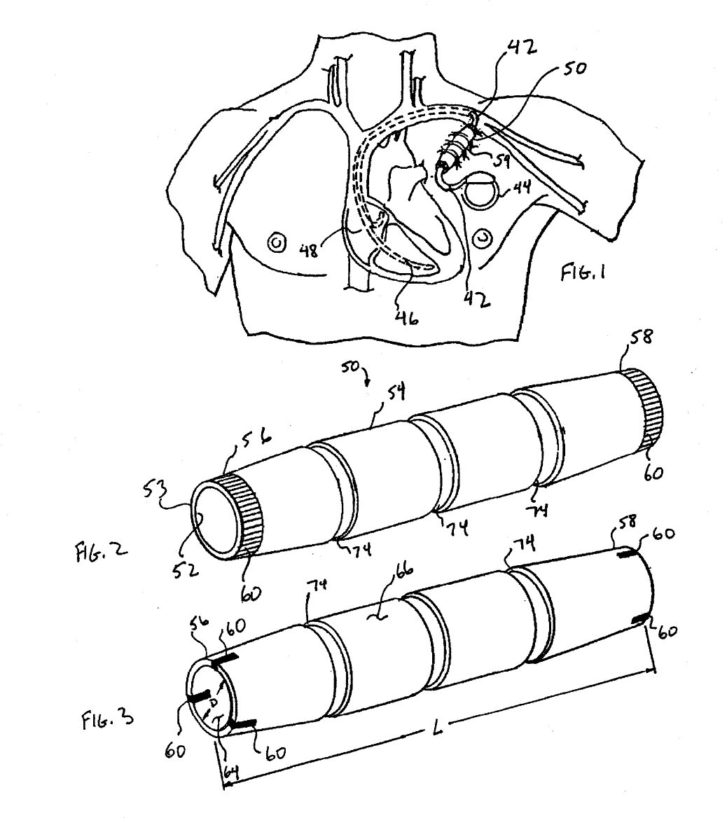

FIG. 1 depicts anchoring sleeve 50 in accordance with the invention, including tube 54 of flexible material with radiopaque material 60 on selected areas of sleeve 54 so that a surgeon may ascertain location and size of sleeve 50.

As shown, sleeve 50 has inner diameter 52 through which lead 42 is placed. Lead 42 extends from implantable device 44 and electrode 46 is placed on the opposite end of lead 42 where electrode 46 and/or a part of lead 42 is secured within a human body, such as within muscle 48 or tissue, including a heart, lung, and the like.

As shown, implantable device 44 is a pacemaker or defibrillator. In some embodiments, implantable device 44 is any other medical device that is implanted in the body, such as a kidney dialysis catheter, vascular shunt, chemotherapy delivery port, and the like. In other embodiments, implantable device 44 is a medical device placed outside the body but where lead 42 and electrode 46 extend into the body. In fact, sleeve 50 is useful in any application where device 44 and/or lead 42 is secured within the body.

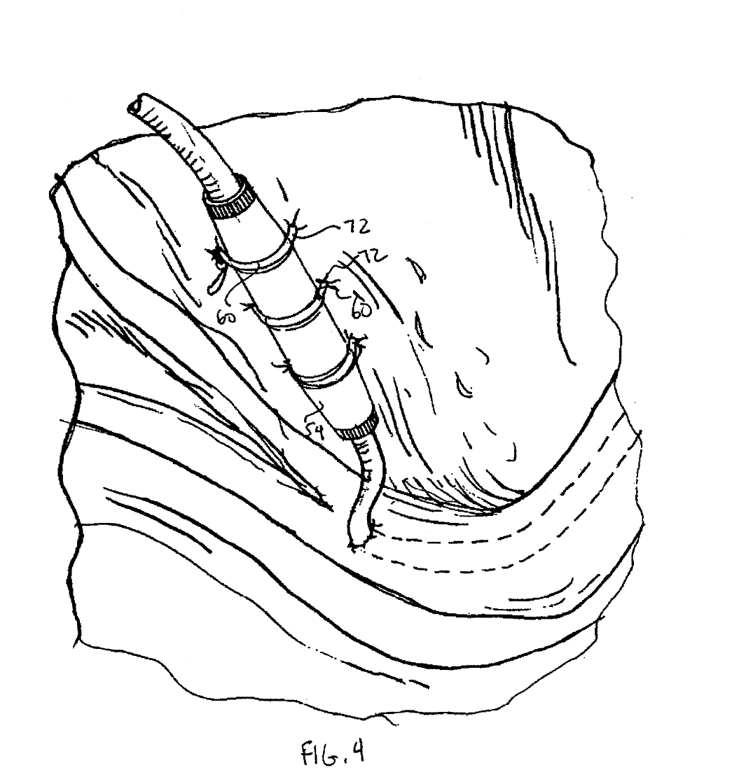

Referring to FIG. 2, sleeve 50 has first end 56 and second end 58. Radiopaque material 60 is placed around each end (circumference of first end 56 and circumference of second end 58) in a shape of a ring so that the surgeon can ascertain, via an instrument for seeing or sensing radiopaque material 60, a length of sleeve 50 as well as a diameter or width of sleeve 50 based on the diameter of the ring of radiopaque material 60 and the distance between the rings of radiopaque material 60 located on first and second ends 56, 58.

FIG. 3 depicts another embodiment where radiopaque material 60 is placed on localized areas of the ends, such as in three locations equidistant apart from one another. In this effort, both length and diameter of sleeve 50 can be ascertained while also providing a savings in amount of radiopaque material 60 used, which may also reduce manufacturing costs. As shown in FIG. 3, length L of sleeve 50 is ascertained by measuring or visually inspecting a distance between first and second ends 56, 58 where radiopaque material 60 are located at each end. Moreover, diameter D or width of sleeve is ascertained since radiopaque material 60 is located at three points, which define an arc, on first end 56 and second end 58.

Hence, radiopaque material 60 is in a first location for indicating length L, where the first location is anywhere on first end 56 and anywhere on second end 58. Radiopaque material 60 is further located in a second location for indicating a diameter D or width of sleeve 50, where the second location is an entire circumference of first end 56 or second end 58, a three point location as shown in FIG. 3, or the equivalent where the diameter of sleeve 50 is indicated.

In another embodiment, the entire sleeve 50 is covered with radiopaque material 60. Radiopaque material 60 is any material applied to sleeve 50 that is not transparent to X-rays or other forms of radiation. Materials that prevent the passage of radiation or electromagnetic radiation are called ‘radiopaque’. In modern medicine, radiopaque usually refers to substances that will not allow x-rays or similar radiation to pass. Various examples of radiopaque material 60 include iodinated contrast (dye) or metal dust (titanium, stainless steel). Radiopaque material 60 is applied through spray on techniques, adhesive, or any other known manners.

As shown in FIG. 4, because sleeve 50 is of a flexible material, stitch 72 compresses sleeve 50 against lead 42 when lead 42 is secured or sewn to chest wall 48. This is advantageous because it is known that sewing stitch 72 directly against lead 42 can damage or shorten the life of lead 42. Therefore, sleeve 50 acts as a protector and reduces the damage to lead 42 from stitch 72 but still permitting stitch 72 to secure lead 42 to chest wall 48.

Sleeve 50 may be sized to fit a variety of leads and the invention envisions sleeve 50 accommodating a variety of different leads from a variety of implantable devices, including varying lengths and diameters.

In some embodiments, length L of sleeve 50 is between approximately 10 mm and approximately 100 mm, inner diameter 64 is less than approximately 50 mm, and outer diameter 66 is between approximately 0.5 mm and approximately 50 mm. In other embodiments, length L of sleeve 50 is between approximately 20 mm and approximately 30 mm, inner diameter 64 is less than approximately 30 mm, and outer diameter 66 is between approximately 2 mm and approximately 5 mm. In further embodiments, length L of sleeve 50 is between approximately 22 mm and approximately 28 mm, inner diameter 64 is less than approximately 10 mm, and outer diameter 66 is between approximately 2 mm and approximately 4 mm.

As shown in FIGS. 1-4, at least one groove 74 is placed in tube 54 for placement of stitch 72, where stitch 72 is used to secure tube 54 and lead 42 to chest wall 48. At least one groove 74 enables a better securement of tube 54 because slippage in an axial direction is minimized, which may permit tube 54 to become dislodged or to shift out of place.

In some of these embodiments, radiopaque material 60 is placed in said at least one groove 74 so that stitch 72 is located. Knowing where stitch 72 is located allows the surgeon to more easily remove tube 54 since removing stitch 72 will allow tube 54 to be removed. Moreover, knowing the quantity of at least one groove 74 gives an indication of the quantity of stitches 72 since there is usually a one to one ratio of groove to stitch. In the event a single stitch is used that is wrapped about tube 54, the quantity of at least one groove 74 indicates the quantity of wraps, or encirclements, of stitch 72 about tube 54. Optionally, stitch 72 includes radiopaque material 60 that coats stitch 72 in addition to or instead of at least one groove 74.

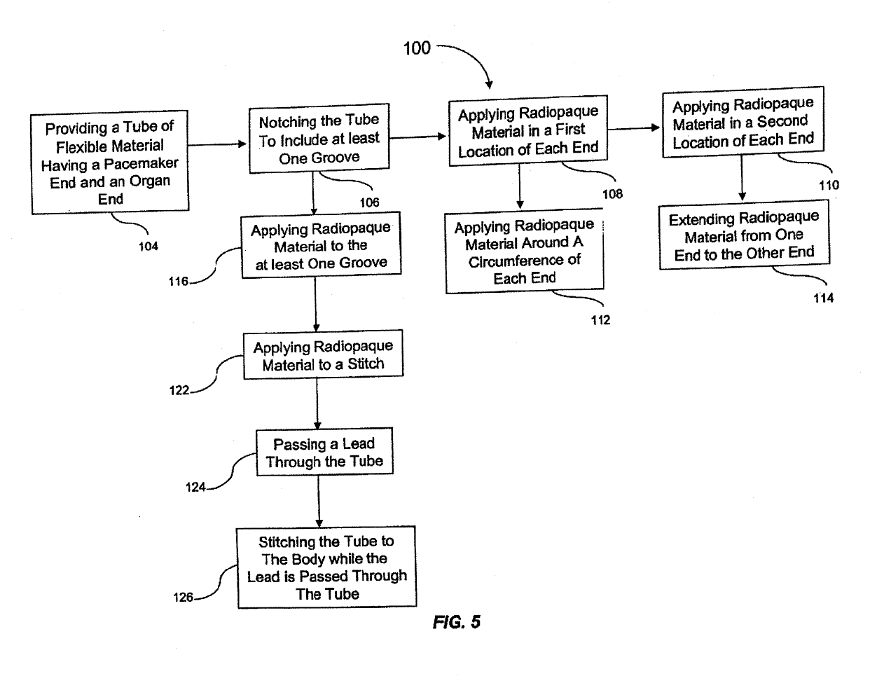

As shown in FIG. 5, method 100 for providing an anchoring sleeve for facilitating attachment of a lead from a pacemaker to a chest wall includes the steps providing 104 a tube of flexible material having a pacemaker end proximate the pacemaker and a chest wall end proximate the chest wall and notching 106 the tube to include at least one groove for placement of a stitch. Method 100 also includes applying 108 a radiopaque material in a first location of each of the pacemaker and chest wall ends for indicating a length of the tube and applying 110 a radiopaque material in a second location of each of the pacemaker and chest wall ends for indicating a width of the tube.

In some embodiments, method 100 also includes applying 112 the radiopaque material around a circumference of the pacemaker and chest wall ends. In other embodiments, method 100 includes extending 114 the radiopaque material from the pacemaker end to the chest wall end and applying 116 radiopaque material in the at least one groove.

In further embodiments, method 100 applies 122 radiopaque material to the stitch, passing 124 a lead through the tube, and stitching 126 the tube to the body while the lead is passed through the tube.

What is claimed is:

1. An anchoring sleeve for a lead of an implantable device, comprising:
a tube being of flexible material having a first end and a second end;
said tube being adapted to permit the lead to extend from the first end to the second end; and
said tube includes a radiopaque material in at least one location.

2. The anchoring sleeve according to claim 1, wherein said at least one location includes a localized area of said first end and said second end.

3. The anchoring sleeve according to claim 1, wherein said at least one location includes a circumference of said first end and said second end.

4. The anchoring sleeve according to claim 3, wherein said at least one location extends from said first end to said second end.

5. The anchoring sleeve according to claim 1, wherein the implantable device is a pacemaker and wherein the lead extends from the pacemaker and through said first and second ends.

6. The anchoring sleeve according to claim 1, further comprising a stitch, wherein said tube compresses to permit said stitch to secure said lead to a chest wall.

7. The anchoring sleeve for according to claim 1, wherein said first end and said second end each includes a radiopaque material in a first location for indicating a length of said tube; and
wherein said first end and said second end each includes a radiopaque material in a second location for indicating a width of said tube.

8. The anchoring sleeve according to claim 7, wherein said tube includes at least one groove for placement of a stitch.

9. The anchoring sleeve according to claim 8, wherein said at least one groove includes radiopaque material.

10. The anchoring sleeve according to claim 8, wherein said stitch includes radiopaque material.

11. An anchoring sleeve for a lead of an implantable device, comprising:
a pacemaker with a lead extending from said pacemaker;
a tube having an inner diameter, a pacemaker end, and a chest wall end;
said tube being of flexible material and adapted to permit said lead extend from said pacemaker end to said chest wall end;
said tube includes at least one groove for placement of a stitch;
said chest wall end and said device end each includes a radiopaque material in a first location for indicating a length of said tube; and
said chest wall end and said device end each includes a radiopaque material in a second location for indicating a width of said tube.

12. The anchoring sleeve according to claim 11, wherein said at least one location includes a circumference of said first end and said second end.

13. The anchoring sleeve according to claim 12, wherein said at least one location extends from said first end to said second end.

14. The anchoring sleeve according to claim 11, wherein said at least one groove includes radiopaque material.

15. The anchoring sleeve according to claim 11, wherein said stitch includes radiopaque material.

16. A method for providing an anchoring sleeve for facilitating attachment of a lead from a pacemaker to a chest wall, comprising the steps of:
providing a tube of flexible material having a pacemaker end proximate the pacemaker and a chest wall end proximate the chest wall;
notching the tube to include at least one groove for placement of a stitch;
applying a radiopaque material in a first location of each of the first and second ends for indicating a length of the tube; and
applying a radiopaque material in a second location of each of the first and second ends for indicating a width of the tube.

17. The method according to claim 16, further comprising the step of applying the radiopaque material around a circumference of the pacemaker and chest wall ends.

18. The method according to claim 16, further comprising the step of extending the radiopaque material from the pacemaker end to the chest wall end.

19. The method according to claim 16, further comprising the step of applying radiopaque material in the at least one groove.

20. The method according to claim 16, further comprising the step of applying includes radiopaque material to the stitch.

21. The method according to claim 16, further comprising the steps of passing a lead through the tube and stitching the tube to the body while the lead is passed through the tube.
